(19) United States
(12) Patent Application Publication
(10) Pub. No.: US 2026/0207770 A1
(43) Pub. Date: Jul. 23, 2026

(54) MEMBRANE COATED NANOPARTICLES FOR MODULATING AN IMMUNE RESPONSE

(71) Applicant: The Regents of the University of Michigan, Ann Arbor, MI (US)

(72) Inventors: Feiran Li, Ann Arbor, MI (US); Lonnie Shea, Ann Arbor, MI (US); Elizabeth Jean Bealer, Ann Arbor, MI (US)

(21) Appl. No.: 19/137,347

(22) PCT Filed: Dec. 19, 2023
(86) PCT No.: PCT/US2023/084747

§ 371 Date: Jun. 10, 2025

Related U.S. Application Data
(60) Provisional application No. 63/434,233, filed on Dec. 21, 2022.

(51) Int. Cl. A61K 47/69; A61K 9/51; A61K 39/00; A61K 39/39; A61K 40/19; A61K 40/22

(52) U.S. Cl. CPC A61K 47/6901; A61K 9/5176; A61K 39/39; A61K 40/19; A61K 40/22; A61K 47/6937; A61K 2039/55555; A61K 2039/577

(57) ABSTRACT

The present disclosure relates to compositions, systems, and methods for modulating immune cell responses in a subject. In particular, the disclosure provides membrane-coated nanoparticles comprising a nanoparticle shell coated with a cell membrane obtained from an antigen presenting cell, and methods of use thereof to modulate an immune response in a sample or a subject.

### PRIORITY STATEMENT

This application claims priority to U.S. Provisional Patent Application No. 63/434,233, filed Dec. 21, 2022, the entire contents of which are incorporated herein by reference for all purposes.

### STATEMENT REGARDING FEDERALLY SPONSORED RESEARCH OR DEVELOPMENT

This invention was made with government support under RO1 AI15678 and RO1 AI48076, awarded by the National Institutes of Health. The government has certain rights in the invention.

### FIELD

The present disclosure relates to compositions, systems, and methods for modulating immune cell responses in a subject. In particular, the disclosure provides membrane-coated nanoparticles comprising a nanoparticle shell coated with a cell membrane obtained from an antigen presenting cell, and methods of use thereof to modulate immune responses in a subject.

### BACKGROUND

The modulation of T cell responses has been a target for treating undesired immune responses, such as autoimmune disease and transplant rejection. Current approaches for modulating T cell responses include pharmaceuticals, such as tacrolimus and sirolimus, or more recently immune checkpoint blockade. However, these immunosuppression strategies typically act through the interaction with the signaling pathways of T cells, which can suppress activity for all T cells and thus make the patient susceptible to opportunistic infections and neoplasia with long term use. Accordingly, what is needed are targeted approaches that target immune cells, such as T cells, in an antigen specific manner while avoiding these harmful side effects.

### SUMMARY

In some aspects, provided herein are membrane-coated nanoparticles. In some embodiments, provided herein are membrane-coated nanoparticles, comprising a nanoparticle core coated with a cell membrane obtained from a cell that contains antigens. In some embodiments, the cell that contains antigens is an antigen presenting cell. In some embodiments, the antigen presenting cell is a regulatory antigen presenting cell or a tolerogenic antigen presenting cell. In some embodiments, the antigen presenting cell is a dendritic cell. In some embodiments, the dendritic cell is a regulatory dendritic cell. In some embodiments, the dendritic cell is a tolerogenic dendritic cell. In some embodiments, the regulatory dendritic cell is a bone marrow-derived regulatory dendritic cell. In some embodiments, the regulatory dendritic cell is an activated bone marrow-derived regulatory dendritic cell. In some embodiments, the cell that contains antigens is a stem cell. In some embodiments, the nanoparticle core comprises poly(lactide-co-glycolide) (PLG).

In some embodiments, the membrane-coated nanoparticle expresses three or more surface markers selected from CD11c, MHC-II, CD80, CD86, CD40, PD-L1, and ICOSL. For example, in some embodiments the membrane-coated nanoparticle expresses five or more of CD11c, MHC-II, CD80, CD86, CD40, PD-L1, and ICOSL. In some embodiments, the membrane-coated nanoparticle expresses each of CD11c, MHC-II, CD80, CD86, CD40, PD-L1, and ICOSL.

In some embodiments, the membrane-coated nanoparticle is substantially spherical in shape. In some embodiments, the membrane-coated nanoparticle is substantially spherical in shape and has an average diameter of about 300 to about 800 nm. In some embodiments, the membrane-coated nanoparticle is substantially spherical in shape and has an average diameter of about 300 to about 500 nm. For example, in some embodiments the membrane-coated nanoparticle has an average diameter of about 360 nm to about 420 nm.

In some embodiments, the membrane-coated nanoparticle further comprises one or more immunomodulators conjugated to the cell membrane. For example, in some embodiments the membrane-coated nanoparticle further comprises one or more immunomodulatory ligands conjugated to the cell membrane.

In some aspects, provided herein are methods of producing membrane-coated nanoparticles. In some embodiments, provided herein is a method of producing a membrane-coated nanoparticle, comprising extracting a cell membrane from a cell that contains antigens, and coating a nanoparticle core with the cell membrane.

In some embodiments, the nanoparticle core comprises poly(lactide-co-glycolide) (PLG). In some embodiments, the cell that contains antigens is an antigen presenting cell. In some embodiments, the antigen presenting cell is a regulatory antigen presenting cell or a tolerogenic antigen presenting cell. In some embodiments, the antigen presenting cell is a dendritic cell. In some embodiments, the dendritic cell is a regulatory dendritic cell. In some embodiments, the dendritic cell is a tolerogenic dendritic cell. In some embodiments, the regulatory dendritic cell is a bone marrow-derived regulatory dendritic cell. In some embodiments, the regulatory dendritic cell is an activated bone marrow-derived regulatory dendritic cell. In some embodiments, the cell that contains antigens is a stem cell.

In some embodiments, the membrane-coated nanoparticles described herein find use in methods of modulating an immune response in a sample or a subject.

In some aspects, provided herein are methods of modulating an immune response in a sample or a subject. In some embodiments, provided herein are methods of modulating an immune response in a sample or a subject comprising contacting the sample or the subject with a membrane-coated nanoparticle described herein. In some embodiments, the immune response comprises a T-cell response, a B-cell response, and/or an NK-cell response. Accordingly, in some embodiments provided herein are methods of modulating a T-cell response, a B-cell response, and/or an NK-cell response in a sample or a subject.

### DESCRIPTION OF THE DRAWINGS

FIGS. 1A-1B show fabrication and characterization of MCNPs. The MCNPs have two parts: membrane extracted from immune cells and PLGA nanoparticle core. MCNPS were fabricated by extracting cell membranes using a filter size of 800 nm, and then co-extruding the extracted cell membranes with the PLGA nanoparticle core using a filter size of 800 nm. FIG. 1A shows MCNPs'z-average diameter (PLGA core: 367.2±23.0 nm; A 20:389.2±22.1 nm; BMDC: 411.8±6.31 nm), and FIG. 1B shows the surface-potential (PLGA core: −29.8±1.13 mV; A 20: −11.6±0.153 mV; BMDC: −11.2±0.555 mV) obtained using dynamic light scattering, n=3.

FIGS. 2A-2C show MCNPs'surface molecules and Coomassie staining compared to BMDC. FIG. 2A and FIG. 2B show representative flow cytometry analysis of surface molecules on BMDC and MCNPs, n=4. All antibody markers gated based on FMO and negative/isotype control with BMDC or MCNPs. FIG. 2C shows Coomassie stain comparison between BMDC MCNPs, BMDC membrane, and BMDC.

FIGS. 3A-3D show in-vitro direct interaction between MCNPs and naïve T cells from 4C or C57BL/6 mice. FIG. 3A and FIG. 3B show the day-3 proliferation (by CFSE dilution) of 4C T cells with BALB/c BMDC or BALB/c BMDC MCNP. FIG. 3C and FIG. 3D show the day-3 proliferation of C57BL/6 T cells with BALB/c BMDC or BALB/c BMDC MCNP, n=6. ***P<0.001, ****P<0.0001, Student's t test.

FIGS. 4A-4D show in-vitro direct interaction between MCNPs and activated T cells from C57BL/6 mice. FIG. 4A is an illustration of timeline and experimental groups of the study. FIG. 4B shows the levels of T cell proliferation from different co-culture conditions, n=4. FIG. 4C shows Propidium iodide and Annexin V staining of T cells co-cultured with experimental conditions or control by flow cytometry analysis, n=4. FIG. 4D shows cytokine concentrations in the co-culture media on Day 3 by ELISA analysis, n=4. *P<0.05, **P<0.01, ***P<0.001, ****P<0.0001, Student's t test.

FIGS. 5A-5C show OVA323-339 antigen presentation by MCNPs pre-or post-fabrication. FIG. 5A is a schematic showing timelines for OVA antigen presentation to OTII CD4 T cells by BMDC MCNP. FIG. 5B and FIG. 5C show OTII CD4 T cell proliferation under varied experimental or control conditions, n=3. ***P<0.001, ****P<0.0001, Student's t test.

FIGS. 6A-6C show tunable MCNPs'surface molecules expressions by different culturing conditions. FIG. 6A shows representative flow cytometry analysis of surface molecules on BMDC and MCNPs, n=4. All antibody markers gated based on FMO and negative/isotype control with BMDC or MCNPs. FIG. 6B shows the levels of T cell proliferation from different co-culture conditions, n=4. FIG. 6C shows cytokine concentrations in the co-culture media on Day 3 by ELISA analysis, n=4. *P<0.05, **P<0.01, ***P<0.001, ****P<0.0001, Student's t test.

FIGS. 7A-7C show biotinylated MCNPs conjugated with streptavidin-FasL protein. FIG. 7A is an illustration of an exemplary MCNP biotinylating process. FIG. 7B and FIG. 7C show Propidium iodide and Annexin V staining of A20 cells co-cultured with free SA-FasL, blank MCNP with SA-FasL, and MCNP-biotin-SA-FasL, n=4 or 6. All conjugated MCNPs were washed three times before co-culturing with A20 cells. ****P<0.0001, Student's t test.

FIGS. 8A-8B show that biodistribution of MCNPs is similar to uncoated NPs. FIG. 8A is a schematic showing MCNPs were delivered intravenously to 4C mice at Day 0 and tissues were harvested 16 hours later. FIG. 8B shows representative images taken 16 hours post intravenous injection using IVIS Living Image software. MCNP and NPs (n=4 and n=3 respectively).

FIG. 9A-9B show that the size of the nanoparticle and the filter used for membrane extrusion and co-coextrusion confer significant differences to the amount of CD80 and MHC-II on the MCNPs produced. NPs of approximately 300 nm and 400 nm were made and co-extruded with cell membranes using 800 nm and 1000 nm filters respectively. Using a smaller NP core (e, g. 300 nm) and a larger filter size for membrane extrusion and membrane and NP co-extrusion (e.g. 1 micron) increases percentage of CD 80 (FIG. 9A) and MHC-II (FIG. 9B) on the MCNPs. (N=3 for each condition).

FIG. 10 is a schematic showing an exemplary fabrication method for MCNPs, determined based upon the data presented in FIG. 9. Suitable antigen-containing cells, in this embodiment BALB/c DCs, are lysed using dounce homegenization in hypotonic solution. After homegenization and centrifugation at speeds reaching 100,000 g, membranes are extruded with suitable filter. The filter used in this exemplary embodiment is a 1000 nm (i.e. 1 micron) filter. Extruded membranes are mixed with blank NPs and co-extruded through a suitable filter to form MCNPs. The filter used in this exemplary embodiment is also a 1000 nm (i.e. 1 micron) filter.

FIG. 11 shows that T cell proliferation increases using the optimized fabrication method of MCNPs. Increased MHC-II and CD80 expression on MCNPs generated using a smaller core (e.g. 300 nm) and a larger filter size (e.g. 1 micron) for extrusion and coextrusion, as shown in FIG. 9A-9B and in the schematic of FIG. 10, corresponds to increase in CFSE+signal indicating greater CD4+ T cell proliferation following co-culture with 4C T cells. More MCNPs can directly communicate with T cells. (Top) N=3 for T cell proliferation percentage after culture for 3 days with blank NPs and MCNPs. (Bottom) representative images of CFSE+ corresponding to T cell proliferation. Data was collected and processed using FlowJo v10 software.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof).

As used herein, the terms “prevent,” “prevention,” and preventing” refer to reducing the likelihood of a particular condition or disease state from occurring in a subject not presently experiencing or afflicted with the condition or disease state. The terms do not necessarily indicate complete or absolute prevention.

The terms “subject” and “patient” are used interchangeably herein and refer to any animal. In some embodiments, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. In some aspects, the human is an adult aged 18 years or older. In some aspects, the human is a child aged 17 years or less.

### DETAILED DESCRIPTION

In some aspects, described herein nanoparticles coated with cell membranes, termed membrane coated nanoparticles (MCNPs). In some aspects, described herein is the use of MCNPs to modulate immune responses. In some embodiments, MCNPs are used to directly modulate T cell responses. For example, in some embodiments, membranes from dendritic cells (DCs) and regulatory dendritic cells (DCregs) are combined with PLG nanoparticles to produce MCNPs that directly modulate T cell responses. MCNPs are shown to have similar membrane characteristics as the DCs from which they were derived. These MCNPs are able to directly engage with and modulate T cell responses in an allogeneic setting. Additionally, cell membranes can be engineered either pre-or post-fabrication to control antigen and costimulatory molecules presentation. The MCNPs thus provide a versatile tool capable of directly engaging T cells to modulate responses.

In some aspects, provided herein are membrane-coated nanoparticles. In some embodiments, provided herein is a membrane-coated nanoparticle comprising a nanoparticle (NP) core coated with a cell membrane obtained from a cell that contains antigens. The terms “nanoparticle core”, “nanoparticle” “NP”, “NP core”, and “core” are used interchangeably herein. In some embodiments, the MCNPs present the antigens directly or semi-directly. In some embodiments, the MCNPs are internalized by antigen presenting cells for indirect antigen presentation. In some embodiments, the cell that contains antigens is an antigen presenting cell (APC). The term “antigen presenting cell” is used herein in the broadest sense and refers to an immune cell that mediates the immune response by processing and presenting antigens for recognition by lymphocytes, such as T-cells. Exemplary antigen presenting cells include macrophages, Langerhans cells, B cells, and dendritic cells. In some embodiments, the antigen presenting cell is a regulatory antigen presenting cell. In some embodiments, the antigen presenting cell is a tolerogenic antigen presenting cell. In some embodiments, the antigen presenting cell is a dendritic cell. In some embodiments, the antigen presenting cell is a bone-marrow derived dendritic cell (BMDC). In some embodiments, the dendritic cell is a regulatory dendritic cell. In some embodiments, the regulatory dendritic cell is a bone marrow-derived regulatory dendritic cell. For example, a regulatory dendritic cell may be differentiated from a bone marrow stem cell. Exemplary methods for differentiating a regulatory dendritic cell from a bone marrow stem cell are described in Example 1. For example, in some embodiments regulatory dendritic cells (DCregs) are obtained from bone marrow stem cells by culturing in a low level of GM-CSF, such as 20 ng/ml. In some embodiments, the regulatory dendritic cell is an activated regulatory dendritic cell. The term “activated” indicates that the expression of cell surface antigens such as MHC-II and co-stimulatory molecules (e.g. CD11c, CD80, CD86, CD40, PD-L1, ICOSL, etc.) is increased (e.g. compared to an inactive cell). For example, regulatory dendritic cells can be activated by culturing cells in a medium comprising one or more suitable stimulatory molecules. For example, regulatory dendritic cells can be activated by culturing cells with a cytokine, such as tumor necrosis factor (TNF). As described in Example 1, in some embodiments activated BMDCs are obtained by introducing lipopolysaccharide and TNF-α to the DCreg culture. In some embodiments, the cell that contains antigens is a stem cell. The term “stem cell” is used herein in the broadest sense and refers to an undifferentiated or partially differentiated cell that can differentiate into various types of cells.

In some embodiments, the nanoparticle core comprises a biodegradable, biocompatible polymer. Suitable biocompatible polymers include, for example, polycaprolactone (PCL), polylactide (PLA), poly(lactide-co-glycolide) (PLGA, also referred to as PLG), polyethylene glycol (PEG), acetalated dextran (Ac-Dex), or copolymers based on methacrylic acid and methylmethacrylate (e.g. Eudragit®). For example, in some embodiments, the nanoparticle core comprises poly(lactide-co-glycolide) (PLG, or PLGA).

The nanoparticle core may be any suitable size and shape. Generally, the nanoparticle core is substantially spherical in shape. In some embodiments, the nanoparticle core is substantially spherical in shape and has an average diameter of about 100 nm to about 700 nm. In some embodiments, the nanoparticle core is substantially spherical in shape and has an average diameter of about 100 nm to about 600 nm. In some embodiments, the nanoparticle core is substantially spherical in shape and has an average diameter of about 100 nm to about 500 nm. In some embodiments, the nanoparticle core is substantially spherical in shape and has an average diameter of about 200 nm to about 600 nm. In some embodiments, the nanoparticle core is substantially spherical in shape and has an average diameter of about 200 nm to about 500 nm. In some embodiments, the nanoparticle core is substantially spherical in shape and has an average diameter of about 200 nm to about 400 nm. In some embodiments, the nanoparticle core is substantially spherical in shape and has an average diameter of about 250 nm to about 350 nm. For example, in some embodiments the nanoparticle core is substantially spherical in shape and have an average diameter of about 200 nm, about 220 nm, about 240 nm, about 260 nm, about 280 nm, about 300 nm, about 320 nm, about 340 nm, about 360 nm, about 380 nm, or about 400 nm. In some embodiments, the nanoparticle core is substantially spherical in shape and has an average diameter of about 300 nm. In some embodiments, the size of the nanoparticle core is selected based upon the size of the filter used for extrusion of cell membranes to produce cell membrane vesicles and/or the size of the filter used for co-extrusion of the cell membrane vesicles and the nanoparticle core.

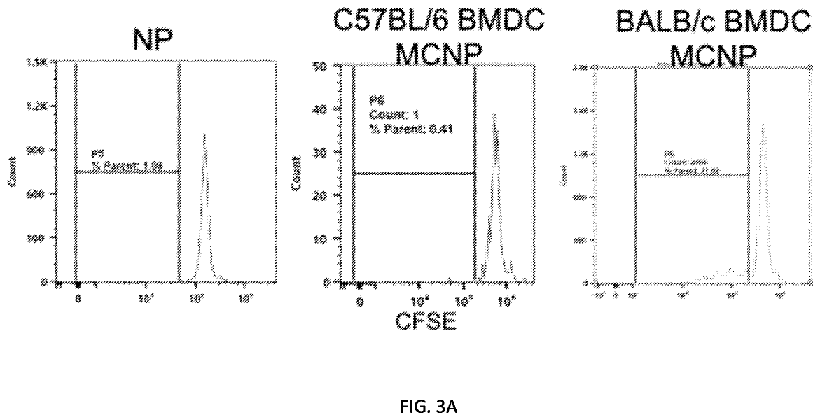

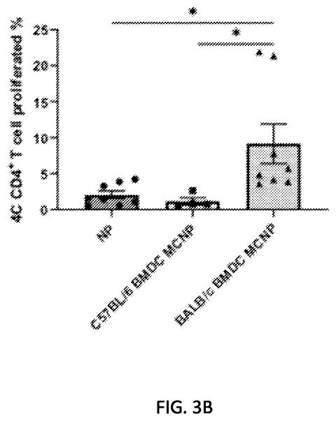

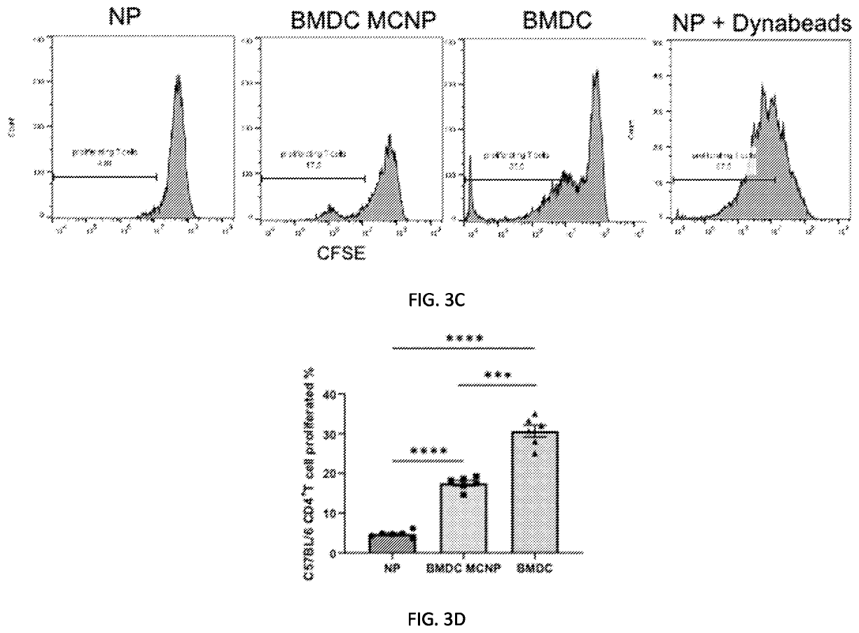

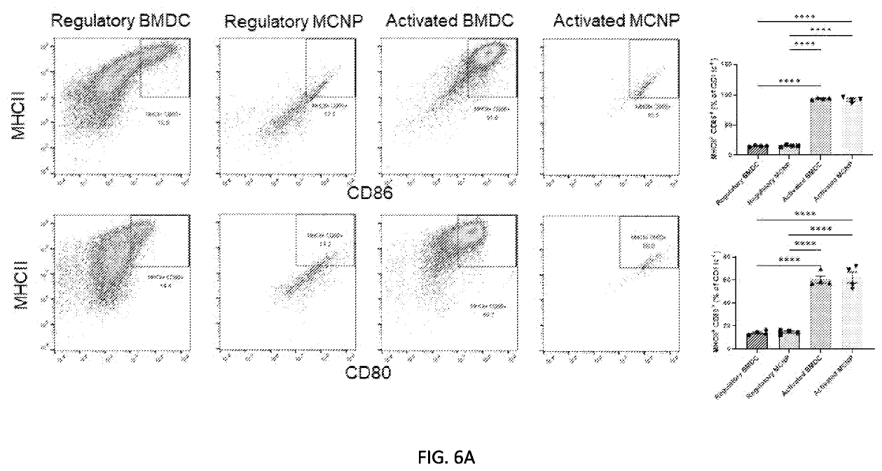

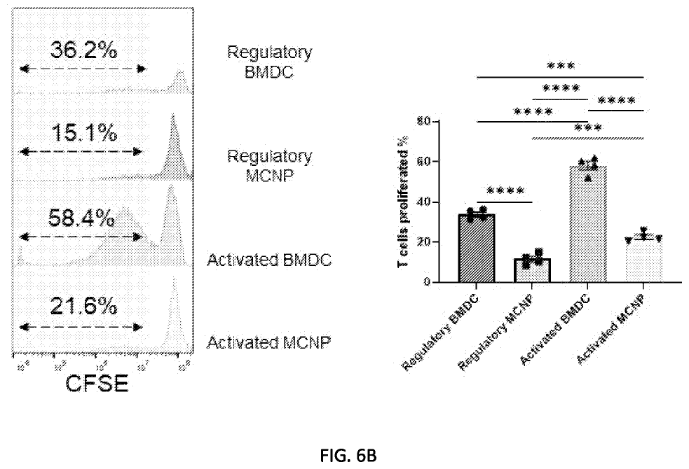

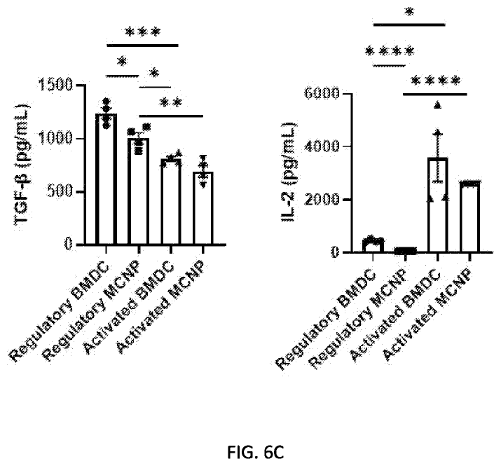

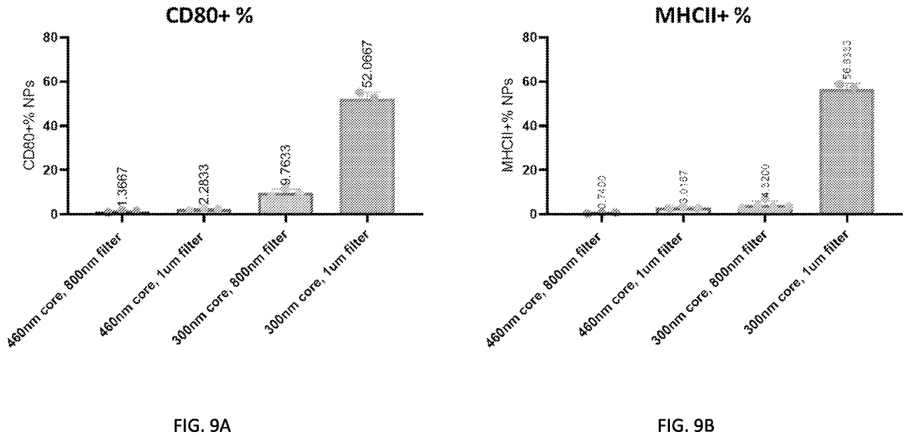

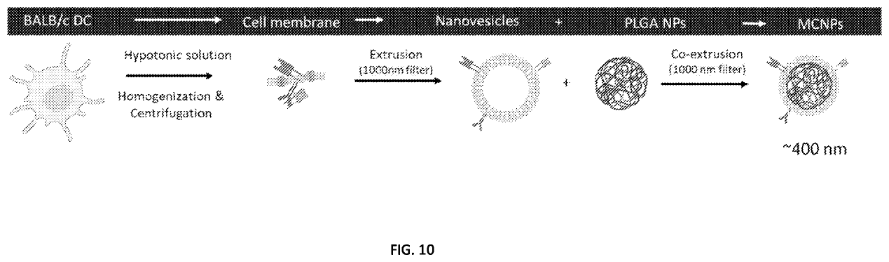

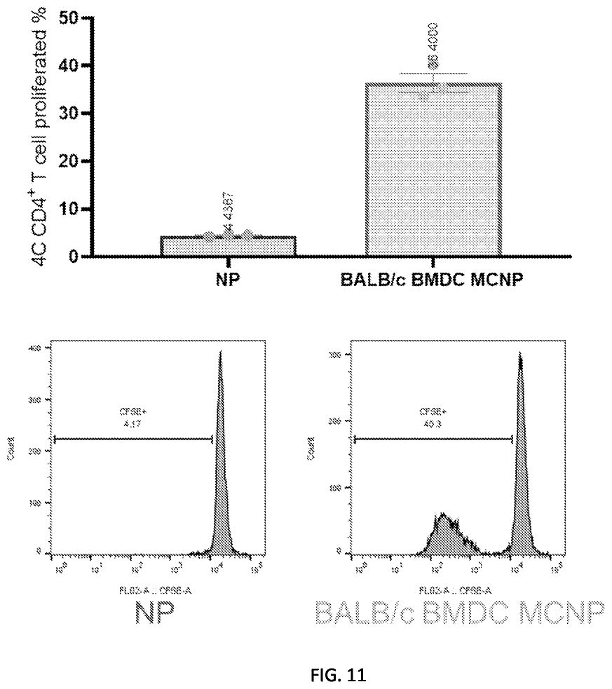

The nanoparticle core is coated with the cell membrane obtained from the cell that contains antigens. For example, the nanoparticle core can be coated with a cell membrane obtained from an antigen presenting cell (e.g. the dendritic cell). The term “coated” as used herein refers to the cell membrane covering a substantial majority of the outer surface of the nanoparticle core, much like an outer shell. However, “coated” does not indicate a complete (i.e. a 100%) coating of the core. For example, a small amount of surface area (e.g. 10% of the surface area or less) of the nanoparticle core may be exposed (e.g. not covered with the cell membrane). In some embodiments, the nanoparticle core is coated with the cell membrane by extrusion. In some embodiments, cell membranes are extracted from a population of cells (e.g. antigen presenting cells such as dendritic cells, regulatory dendritic cells, activated dendritic cells, or stem cells) by lysing the cells and centrifugation to separate the cell membranes. In some embodiments, following extraction of cell membranes, cell membrane vesicles are generated. For example, in some embodiments cell membranes are physically extruded through a suitable membrane for a suitable number of passes to generate cell membrane vesicles. The membrane used for extrusion is also referred to as a “filter”. In some embodiments, the cell membrane vesicles are then coated onto the nanoparticle core, such as by co-extruding the cell membrane vesicles and nanoparticle cores through a suitable membrane. The membrane used for co-extrusion is also referred to herein as a “filter”. The nanoparticle core being coated with a cell membrane is inclusive of the nanoparticle core being coated with a cell membrane vesicle, such as a cell membrane vesicle obtained by extruding a cell membrane through a suitable filter. The experiments demonstrated herein indicate that the size of the nanoparticle core and the size of the filters used for extrusion/co-extrusion can convey significant differences to the resulting MCNP in terms of surface expression of markers and T-cell proliferation following engagement with the MCNPs. For example, the results presented in FIG. 9 demonstrate that a relatively small nanoparticle core size in conjunction with a relatively large filter size results in MCNPs with increased expression of CD80 and MCH-II and increased T-cell proliferation following co-culture with T-cells. Specifically, the results in FIG. 9A and FIG. 9B demonstrate that a nanoparticle core size of 300 nm in conjunction with a filter size of 1 micron (e.g., for the filters used both for extrusion of the cell membranes to produce the cell membrane vesicles, and for co-extrusion of the cell membrane vesicles with the nanoparticle core to coat the nanoparticle core) resulted in a significant increase in the number of CD80+ and MCH-II+ MCNPs produced compared to those produced using a nanoparticle core size of 300 nm in conjunction with an 800 nm filter and those produced using a nanoparticle core size of 460 nm in conjunction with either a 1 micron filter or an 800 nm filter. Moreover, these MCNPs produced by this optimized method result in increased T-cell proliferation following co-culture with 4C T cells (see FIG. 11) compared to MCNPs produced using a smaller filter size (e.g. 800 nm, as shown in FIG. 3B). This increased T-cell proliferation using the optimized method based upon NP core size and filter size was also increased compared to T-cell proliferation seen using MCNPs produced with cell membranes obtained from activated BMDCs (see FIG. 6B).

In some embodiments, the cell membranes are physically extruded through a polycarbonate filter, thereby producing cell membrane vesicles which are subsequently used to coat a nanoparticle core. In some embodiments, the cell membranes are physically extruded through a filter having a pore size of greater than about 600 nm. In some embodiments, the cell membranes are physically extruded through a filter having a pore size of at least about 600 nm to about 1400 nm. In some embodiments, the cell membranes are physically extruded through a filter having a pore size of at least about 800 nm to about 1200 nm. In some embodiments, the cell membranes are physically extruded through a filter having a pore size of at least about 600 nm, at least about 620 nm, at least about 640 nm, at least about 660 nm, at least about 680 nm, at least about 700 nm, at least about 720 nm, at least about 740 nm, at least about 760 nm, at least about 780 nm, at least about 800nm, at least about 820nm, at least about 840 nm, at least about 860 nm, at least about 880 nm, at least about 900 nm, at least about 920 nm, at least about 940 nm, at least about 960 nm, at least about 980 nm, at least about 1000 nm, at least about 1020 nm, at least about 1040 nm, at least about 1060 nm, at least about 1080 nm, at least about 1100 nm, at least about 1120 nm, at least about 1140 nm, at least about 1160 nm, at least about 1180 nm, at least about 1200 nm, at least about 1220 nm, at least about 1240 nm, at least about 1260 nm, at least about 1280 nm, at least about 1300 nm, at least about 1320 nm, at least about 1340 nm, at least about 1360 nm, at least about 1380 nm, or about 1400 nm.

In some embodiments, the cell membranes after extrusion (e.g. the cell membrane vesicles) and the nanoparticles are subsequently co-extruded through a polycarbonate filter, thereby producing the MCNPs. In some embodiments, the cell membrane vesicles and the NPs are co-extruded through a filter having a pore size of greater than about 600 nm. In some embodiments, the cell membrane vesicles and the NPs are co-extruded through a filter having a pore size of at least about 600 nm to about 1400 nm. In some embodiments, the cell membrane vesicles and the NPs are co-extruded through a filter having a pore size of at least about 800 nm to about 1200 nm. In some embodiments, the cell membrane vesicles and the NPs are co-extruded through a filter having a pore size of at least about 600 nm, at least about 620 nm, at least about 640 nm, at least about 660 nm, at least about 680 nm, at least about 700 nm, at least about 720 nm, at least about 740 nm, at least about 760 nm, at least about 780 nm, at least about 800nm, at least about 820nm, at least about 840 nm, at least about 860 nm, at least about 880 nm, at least about 900 nm, at least about 920 nm, at least about 940 nm, at least about 960 nm, at least about 980 nm, at least about 1000 nm, at least about 1020 nm, at least about 1040 nm, at least about 1060 nm, at least about 1080 nm, at least about 1100 nm, at least about 1120 nm, at least about 1140 nm, at least about 1160 nm, at least about 1180 nm, at least about 1200 nm, at least about 1220 nm, at least about 1240 nm, at least about 1260 nm, at least about 1280 nm, at least about 1300 nm, at least about 1320 nm, at least about 1340 nm, at least about 1360 nm, at least about 1380 nm, or about 1400 nm.

In some embodiments, the membrane-coated nanoparticle expresses three or more surface markers selected from CD11c, MHC-II, CD80, CD86, CD40, PD-L1, and ICOSL. In some embodiments, the membrane-coated nanoparticle expresses five or more of CD11c, MHC-II, CD80, CD86, CD40, PD-L1, and ICOSL. In some embodiments, the membrane-coated nanoparticle expresses each of CD11c, MHC-II, CD80, CD86, CD40, PD-L1, and ICOSL.

In some embodiments, the membrane-coated nanoparticle is substantially spherical in shape. In some embodiments, the membrane-coated nanoparticle is substantially spherical in shape and has an average diameter of about 50 nm larger than the diameter of the nanoparticle core used to produce the MCNP. In some embodiments, the MCNP is substantially spherical in shape and have an average diameter of about 150 nm to about 800 nm. In some embodiments, the MCNP is substantially spherical in shape and have an average diameter of about 200 nm to about 800 nm. In some embodiments, the MCNP is substantially spherical in shape and have an average diameter of about 300 nm to about 800 nm. For example, in some embodiments the membrane-coated nanoparticle is substantially spherical in shape and has an average diameter of about 300 nm, about 310 nm, about 320 nm, about 330 nm, about 340 nm, about 350 nm, about 360 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm, about 500 nm, 510 nm, about 520 nm, about 530 nm, about 540 nm, about 550 nm, about 560 nm, about 570 nm, about 580 nm, about 590 nm, about 600 nm, about 610 nm, about 620 nm, about 630 nm, about 640 nm, about 650 nm, about 660 nm, about 670 nm, about 680 nm, about 690 nm, about 700 nm, 710 nm, about 720 nm, about 730 nm, about 740 nm, about 750 nm, about 760 nm, about 770 nm, about 780 nm, about 790 nm, or about 800 nm. In some embodiments, the average diameter is about 300 nm to about 500 nm. In some embodiments, the average diameter is about 300 nm to about 450 nm. In some embodiments, the average diameter is about 300 nm to about 425 nm. In some embodiments, the average diameter is about 360 nm to about 420 nm. For example, in some embodiments, the membrane-coated nanoparticle has an average diameter or about 350 nm, about 360 nm, about 370 nm, about 380 nm, about 390 nm, about 400 nm, about 410 nm, about 420 nm, about 430 nm, about 440 nm, or about 450 nm. In some embodiments, the membrane-coated nanoparticle has an average diameter of about 400 nm to about 420 nm. In some embodiments, the membrane-coated nanoparticle has an average diameter of about 400 nm, about 405 nm, about 410 nm, about 415 nm, or about 420 nm.

In some embodiments, the membrane-coated nanoparticle further comprises one or more immunomodulators conjugated to the cell membrane. The term “immunomodulator” is used in the broadest sense and refers to any agent that stimulates or suppresses the immune system.

Suitable immunomodulators include, for example, antibodies, cytokines, ligands, etc. In some embodiments, the membrane-coated nanoparticle further comprises an immunomodulatory ligand conjugated to the cell membrane. Any suitable method may be used to conjugate the immunomodulator to the cell membrane, including covalent interactions or non-covalent interactions. For example, in some embodiments the cell membrane is biotinylated and an avidin or streptavidin moiety can be attached to the immunomodulator (e.g. ligand), thereby permitting conjugation of the immunomodulator (e.g. the ligand) to the cell membrane via non-covalent interactions between the streptavidin (or avidin) and the biotin in the cell membrane. In some embodiments, more than one immunomodulator is conjugated to the cell membrane of a given membrane-coated nanoparticle.

In some aspects, provided herein are compositions. In some embodiments, provided herein is a composition comprising a plurality of membrane-coated nanoparticles. In some embodiments, the composition comprises a plurality of membrane-coated nanoparticles, wherein a first subset of the plurality of nanoparticles comprise cell membranes obtained from a first type of cell and a second subset of the plurality of nanoparticles comprise cell membranes obtained from a second type of cell. In some embodiments, a first subset of the plurality of nanoparticles comprise cell membranes obtained from a first type of antigen presenting cell and a second subset of the plurality of nanoparticles comprise cell membranes obtained from a second type of antigen presenting cell. In some embodiments, a first subset of the plurality of nanoparticles comprise cell membranes obtained from a first type of stem cell and a second subset of the plurality of nanoparticles comprise cell membranes obtained from a second type of stem cell. In some embodiments, a first subset of the plurality of nanoparticles comprise cell membranes obtained from a stem cell and a second subset of the plurality of nanoparticles comprise cell membranes obtained from an antigen presenting cell. In some embodiments, the composition comprises a first subset of the plurality of nanoparticles comprising a first immunomodulatory agent (e.g. conjugated to the cell membrane), and a second subset of the plurality of nanoparticles comprising a second immunomodulatory agent.

In some aspects, provided herein are methods for modulating an immune response in a sample or a subject. Modulating an immune response may comprise modulating a T-cell response, a B-cell response, and/or an NK-cell response in the sample or subject. For example, in some embodiments provided herein are methods for modulating a T-cell response in a sample or a subject. The term “modulating” when used in reference to a T-cell response is used in the broadest sense and refers to many different means by which a T-cell response can be modulated. For example, modulating a T-cell response may indicate promoting an antigen-specific response in the sample or subject, differentially activating T-cells in the sample or subject, inducing or increasing T-cell proliferation in the sample or the subject, or inducing apoptosis in the sample or subject. In some embodiments, provided herein is a method for modulating a T-cell response in a sample or a subject, comprising contacting the sample or the subject with a membrane-coated nanoparticle or a composition as described herein. In some embodiments, the sample comprises T-cells. In some embodiments, the sample comprises T-cells and the sample is obtained from a subject.

### EXAMPLE 1

The direct modulation of T cell responses has the potential to modulate undesired immune responses including, autoimmune disease, and allogeneic cells transplantation. Described herein is the design of nanoparticles that can directly interact and modulate T cells by coating the membranes from antigen presenting cells (APCs) onto nanoparticles to form membrane-coated nanoparticles (MCNP). Proteins within the membranes of the APCs, such as MHC class II and co-stimulatory factors, were effectively transferred to the MCNP. Using alloreactive T cell models, MCNP derived from allogeneic dendritic cells were able to stimulate proliferation, which was not observed with membranes from syngeneic dendritic cells and influenced cytokine secretion. Furthermore, engineering of the membranes either on the dendritic cells or post-fabrication of MCNP is described herein. Engineered membranes could be generated to promote antigen specific responses, to differentially activate T cells, or to directly induce apoptosis. Collectively, MCNPs represent a tunable platform that can directly interact with and modulate T cell responses.

### Results

### NP Characterization

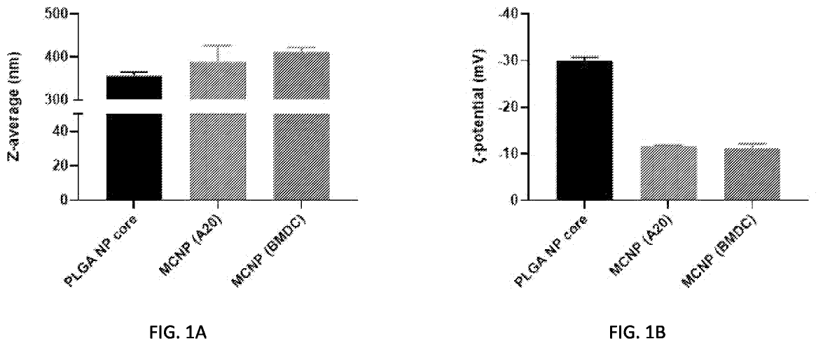

NPs were characterized prior to and following co-extrusion of the NPs with cell membranes extracted in hypotonic solution to form the MCNP. The PLG NPs have a mean diameter of 356.6±8.52 nm and a zeta potential of −29.9±0.802 mV (FIG. 1A, B). The extrusion process produced spherical MCNPs with a mean diameter of 389.2±22.1 nm and zeta potential −11.6±0.153 mV when using A20 membranes or a mean diameter of 411.8±6.31 nm and ζ-potential −11.2±0.555 mV when using bone marrow-derived dendritic cells (BMDCs) membranes. The z-average size of MCNP is overall 50 nm larger compared to PLG NP cores while the surface ζ-potential of MCNPs is 20 mV greater compared to the PLG cores. The increases in both z-average size and ζ-potential suggested the successful coating of the cell membrane on the PLGA NPs.

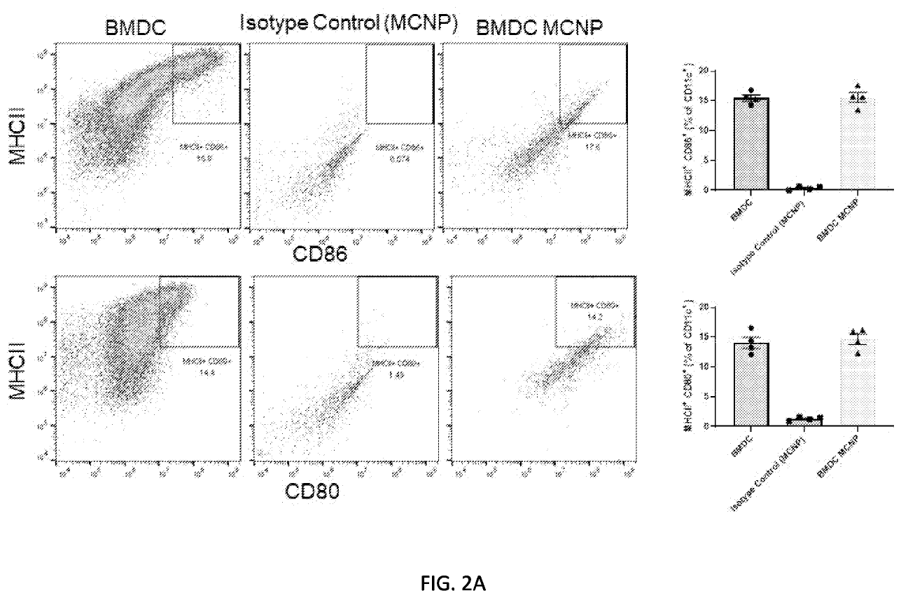

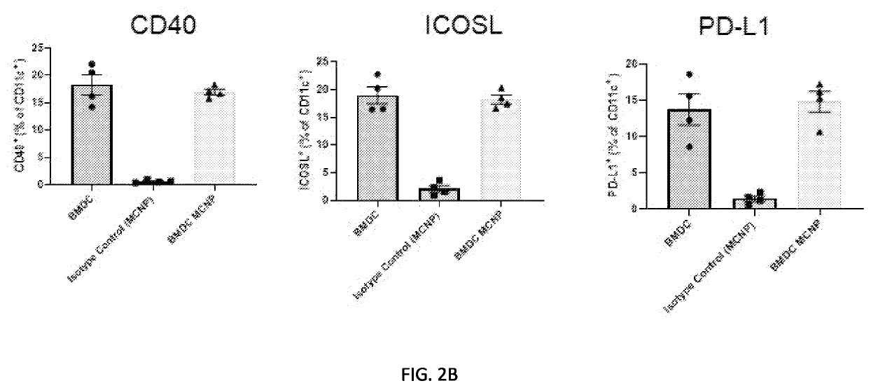

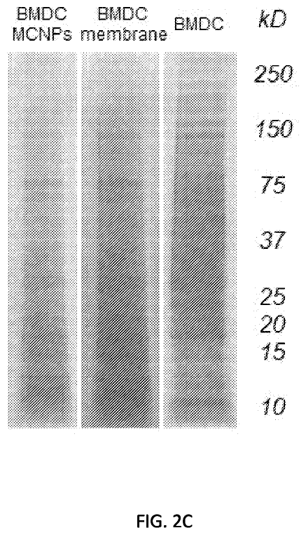

The surface proteins present on the MCNPs were next characterized relative to the donor cell membranes. Regulatory DCs (DCregs) were differentiated from mouse bone marrow stem cells with a low level of GM-CSF at 20 ng/ml. Analysis initially focused on multiple molecules involved in BMDC function and their communication with T cells, namely CD11c, MHCII, CD86, CD80, CD40, ICOSL, and PD-L1. These proteins were present on both BMDCs and the MCNPs, indicating the transfer of membranes and associated proteins to the NPs (FIG. 2A, B). Samples were gated on the CD11c⁺ population before being quantified for the corresponding surface markers as CD11c is considered the most widely used defining marker for dendritic cells. BMDC-derived MCNPs have a similar surface molecule distribution as the DCregs. Coomassic stain further confirmed the membrane molecule/protein transfer from BMDCs to MCNPs. MCNPs retained a similar distribution of protein bands with a mass from 250 kDa to 10 kDa following separation by gel electrophoresis (FIG. 2C).

### Allo-Specific T Cell Responses to MCNPs

The ability of MCNPs to generate antigen-specific T cell responses was investigated using 4C mice transgenic for a T-cell receptor directly recognizing BALB/c MHC class II I-A molecule. BALB/c DCs were differentiated into DCregs with a low level of GM-CSF and used to generate the MCNPs. The antigen specificity was analyzed initially using BALB/c BMDC as stimulators for 4C T cells. The 4C splenocytes labeled with an intracellular fluorescent dye, carboxyfluorescein succinimidyl ester (CFSE), showed robust proliferation when cocultured with MCNPs derived from BALB/c BMDC as compared with unstimulated control with PLG NP cores (FIG. 3A, B). MCNPs generated using syngeneic C57BL/6 DCs membrane did not generate a proliferative response in 4C T cells, further demonstrating antigen-specific nature of 4C proliferation using BALB/c MCNPs.

Allogeneic interactions between MCNPs and T cells were further investigated using BALB/c cells as stimulators and C57BL/6 CD4 T cells as responders, an allogeneic system with an estimated alloreactive repertoire of 10% to 40%. T cell proliferation was increased relative to the negative control for both the BMDC and the MCNP (FIG. 3C, D). The proliferative response of T cells to the MCNP was less than that obtained with the BMDCs. Note that the positive control of T cell stimulation by Dynabeads induced proliferation in more than 65% of T cells, which exceeds the proliferation from BMDCs and is consistent with only a fraction of the C57BL/6 T cells having alloreactivity. These data collectively support the ability of MCNP to promote naïve T cell activation through direct donor-specificity.

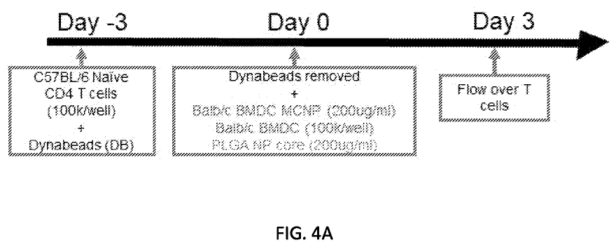

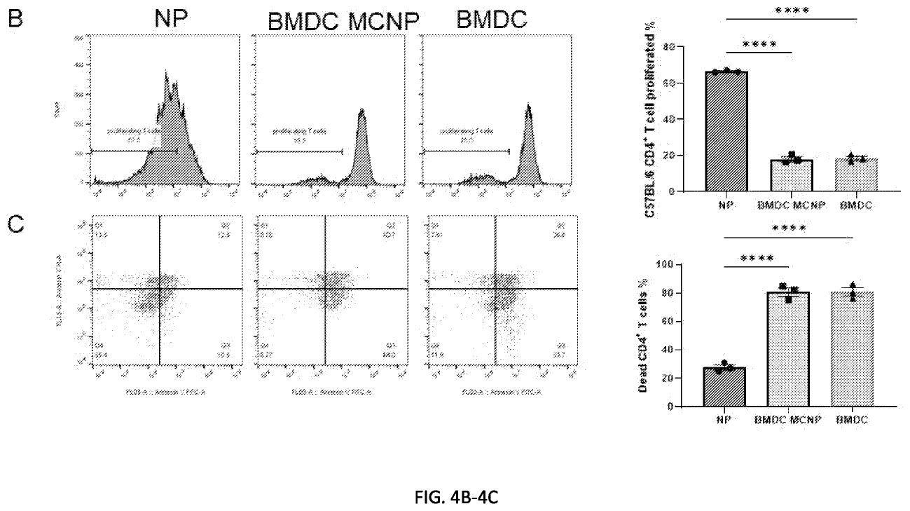

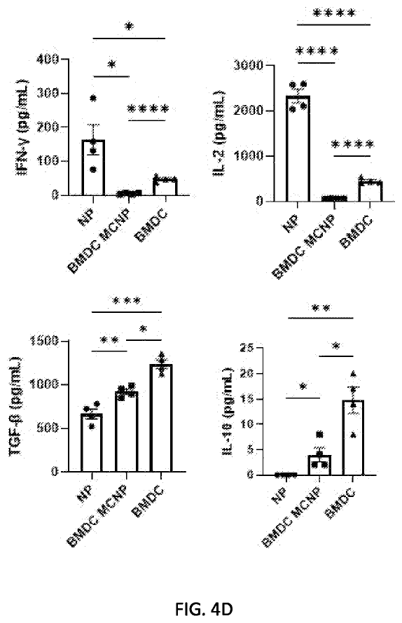

It was next evaluated whether the fabricated MCNPs can interact with the activated CD4 T cells. T cells were activated with Dynabeads that were removed prior to co-culture (FIG. 4A). T cells co-cultured with the NP cores had a robust T cell proliferation, consistent with the activated phenotype induced with Dynabeads. However, the activated T cells co-cultured with DCreg-derived MCNPs had reduced T cell proliferation, where only 16.3% of the population proliferated as compared to 67.5% from NP group (FIG. 4B). A high ratio of cell death, indicated by Annexin V+population, was identified in the co-culture with MCNPs (FIG. 4C). Cytokine levels in the Day 3 co-culture media were measured to further analyze the T cell responses. Interestingly, the pro-inflammatory cytokines, IFN-γ and IL-2, were significantly reduced in the co-culture system with MCNPs, while the anti-inflammatory cytokines, TGF-β and IL-10, were significantly elevated in the MCNPs co-culture (FIG. 4D). Overall, the data support that MCNPs communicate with the activated T cells and influence responses such as proliferation and cytokine secretion.

### Controlled Antigen Loading of MHC Class II on MCNPs

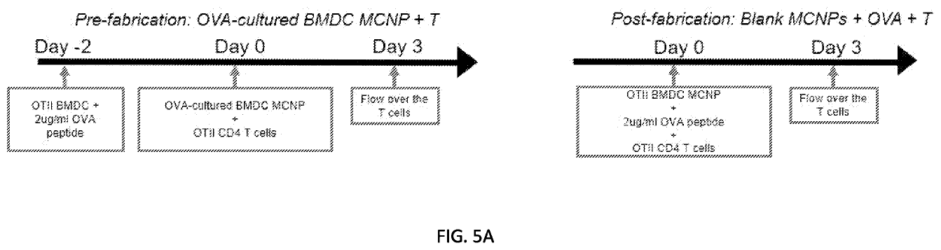

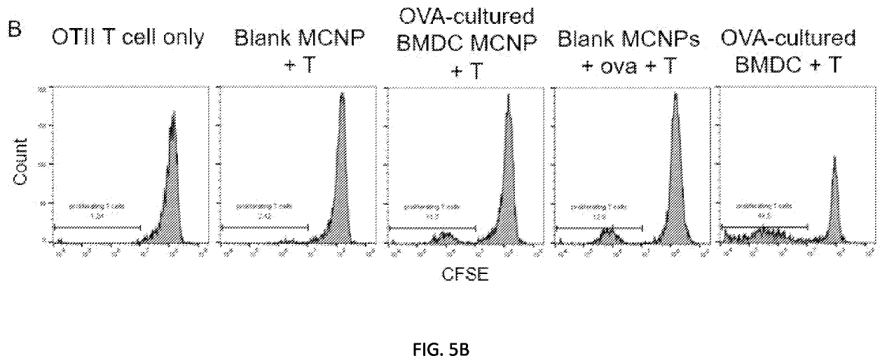

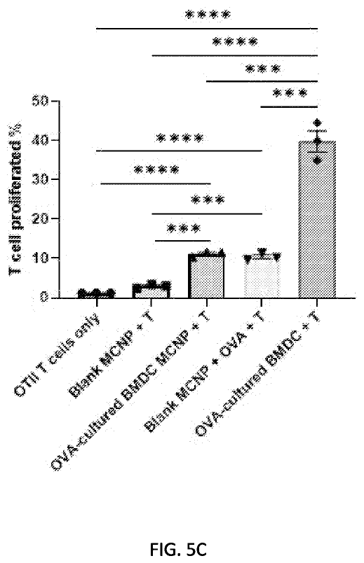

It was next investigated whether the antigen that is presented on the MCNPs can be controlled, for which we used CD4 T cells from OT-II mice that primarily recognize ovalbumin peptide residues 323-339 (OVA₃₂₃₋₃₃₉) when presented by the MHC class II molecule (FIG. 5A). Two approaches for loading of OVA323-339 onto the MHCII were tested. The peptide was incubated with the BMDCs prior to fabrication of the MCNPs, or the MCNPs were prepared and subsequently loaded MHC II with the peptide. OVA T cells had proliferation above the unstimulated controls (OTII T cells only and OTII T cells with blank MCNPs) for both methods of OVA loading onto the MHC. Significantly higher T cell proliferation was found in OVA-cultured BMDC MCNPs compared with those co-cultured with non-OVA added blank BMDC MCNPs (FIG. 5B, C), suggesting direct recognition of the OVA/MHC II complex. Interestingly, no significant difference was observed between two OVA peptide binding methods.

### Engineering MCNPs Through BMDC Culture Conditions

Further engineering of the cell membranes was conducted, where the expression of MHC II and co-stimulatory factors of BMDCs was modulated. DCregs were obtained as described above from mouse bone marrow stem cells with a low level of GM-CSF. The activated BMDCs were obtained by introducing 1 ug/ml lipopolysaccharide and 20 ng/ml TNF-α to the DCreg culture. The activation of BMDCs was confirmed by the increased expressions in MHC-II and co-stimulatory molecules on the activated BMDCs and MCNPs (FIG. 6A) relative to the DCregs and their MCNPs, respectively. Co-culture of the activated BMDC membrane-coated nanoparticles induced greater T cell proliferation compared to the co-culture with regulatory DCs or the associated MCNPs. (FIG. 6B). A decreased production of TGF-β and an increased level of IL-2 were observed for culture with the activated BMDCs or associated MCNPs relative to the regulatory DCs and associated MCNPs (FIG. 6C), further supporting that BMDC culture can be a tool to engineer MCNPs to influence T cell responses.

### Direct Modification of MCNPs to Influence Cell Responses

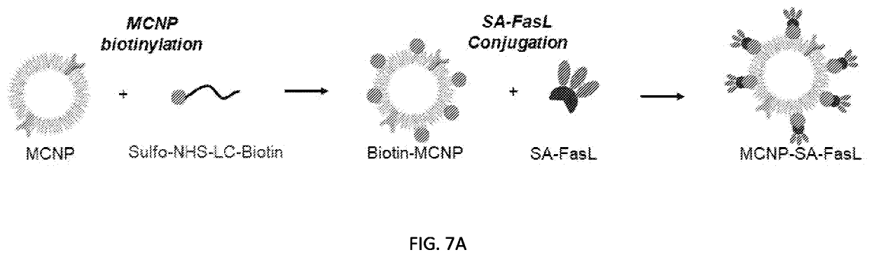

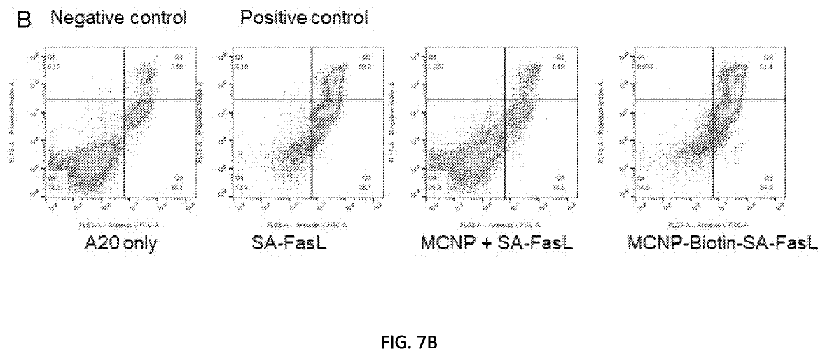

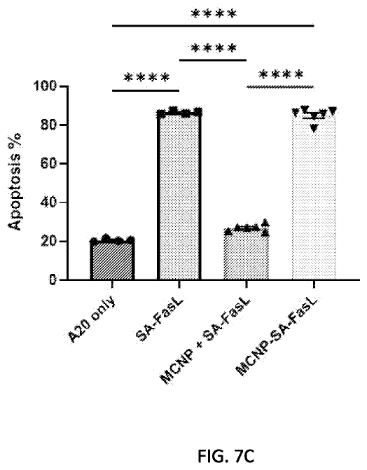

The potential of directly conjugating immunomodulators to the cell membranes post-fabrication was next investigated. MCNPs were produced and subsequently biotinylated to allow for conjugation of streptavidin-Fas ligand (SA-FasL), a novel form of Fas ligand with robust apoptotic activity on Fas receptor positive lymphocytes (FIG. 7A) (Biomaterials. 2019 February:192:271-281. doi: 10.1016/j.biomaterials.2018.11.015. Epub 2018 Nov. 13.). SA-FasL conjugated MCNPs were washed to remove any unbound SA-FasL before co-culturing with A20 cells, a B cell line that expresses Fas. The MCNP-Biotin-SA-FasL induced substantial apoptosis of A20 cells compared to negative control and MCNP+SA-FasL condition, in which MCNP was not biotinylated and thus did not supporting binding of SA-FasL that was likely removed with the washing step (FIG. 7B, C). Collectively, these data demonstrate that MCNPs can be functionalized post-fabrication.

### Biodistribution of MCNPs is Similar to Nanoparticles

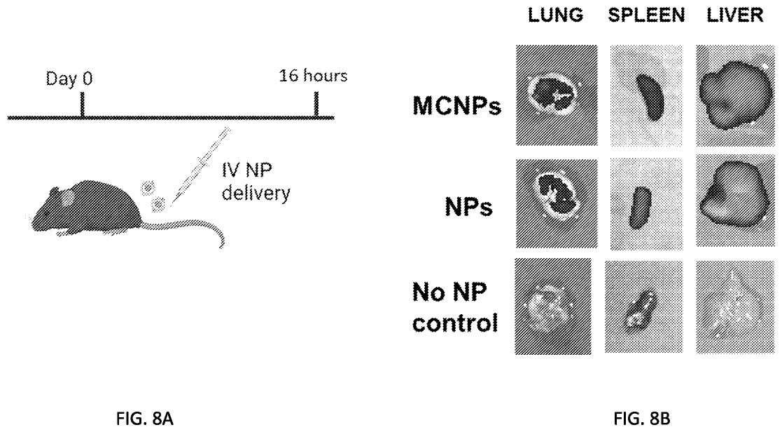

To determine whether membrane coating of nanoparticles influences biodistribution, MCNPs or NPs were delivered intravenously to 4C mice at Day 0 and tissues were harvested 16 hours later. FIG. 8B shows representative images taken 16 hours post intravenous injection using IVIS Living Image software. MCNP and NPs (n=4 and n=3 respectively). As shown, biodistribution of MCNPs is similar to NPs.

### Optimization of MCNP Production

The size of the nanoparticle used for production of MCNPs along with the size of the filter used for extrusion/co-extrusion was varied to optimize the resulting MCNPs. Various sizes of the nanoparticle (NP) core were tested, including 300 nm core and a 460 nm core, in combination with various filter sizes for extrusion of the cell membranes (e.g. from the cell containing antigens) and for co-extrusion of the NPs with the cell membranes. Results are shown in FIG. 9A-9B. The size of the nanoparticle and the filter used for membrane extrusion and co-coextrusion was found to confer significant differences to the amount of CD80 and MHC-II on the MCNPs produced. NPs of approximately 300 nm and 400 nm were made and co-extruded with membranes using 800 nm and 1000 nm filters respectively. Using a smaller NP core (e, g. 300 nm) and a larger filter size for membrane extrusion and membrane and NP co-extrusion (e.g. 1 micron) increases percentage of CD80 (FIG. 9A) and MHC-II (FIG. 9B) on the MCNPs. (N=3 for each condition, Graphs made in GraphPad Prism). Accordingly, an NP core size of about 300 nm in combination with a 1 micron filter was determined to result in improved MCNPs. Moreover, as shown in FIG. 11, these improved MCNPs resulted in increased T cell proliferation following co-culture with BALB/c derived bone marrow dendritic cells. See for example, FIG. 11, which shows about 36% 4C CD 4+ T cells proliferated compared to about 10% proliferation shown in FIG. 3B using MCNPs produced using an 800 nm membrane for extrusion and co-extrusion.

### Discussion

Provided herein are membrane-coated nanoparticles (MCNPs) as a synthetic platform for antigen-specific interactions with T cells. DC therapy modulating T cell responses represents a cell-based approach for antigen-specific modulation of T cells. Upon direct contact between DCs and T cells, the specific combination of MHC and co-stimulatory factors can either induce effector T cell responses or suppress T cell activation. Harnessing the potential for DCs for immunotherapy is challenging due to the likelihood that the cells will encounter alternative signals after delivery that may alter their phenotype and could exacerbate rather than ameliorate pathology. ECDI-fixed splenocytes can induce antigen-specific responses for autoimmune disease and allogeneic transplants. However, limited availability of donor cells and regulatory hurdles are problematic for these therapies.

The MCNPs described herein directly modulate T cell responses. Moreover, MCNPs are not prone to phenotype changes after production, and can manufactured with precise specifications and stored for long periods, enhancing their translatability. Accordingly, the MCNPs described herein represent an effective and feasible alternative to DC-based therapies.

MCNPs are similar in composition to the bone marrow-derived dendritic cells (BMDCs) from which the membranes were derived. The fabricated MCNPs are spherical structure with a diameter of approximately 400 nm, which are substantially smaller than the typical BMDCs with dimension ranging from 15 to 20 μm (Szakal, A. K., Gieringer, R. L., Kosco, M. H. & Tew, J. G. Isolated Follicular Dendritic Cells: Cytochemical Antigen Localization, Nomarski, SEM, and TEM Morphology. J. Immunol. 134, (1985)). As a subset of the APC population, immature BMDCs are characterized by their positive expression in CD11c and MHC-II and low to moderate expression in co-stimulatory molecules such as CD80, CD86, CD40, PD-L1, and inducible costimulatory molecule ligand (ICOSL). These seven surface molecules were analyzed on the fabricated MCNPs, and their levels resemble the biodistribution of those found on the granulocyte-macrophage colony stimulating factor induced immature BMDCs. The fluorescence intensity of the surface molecules differed between BMDCs and MCNPs, which likely results from the differences in the relative surface area of cells and nanoparticles. The Coomassie staining further confirms the similarity between the protein composition between MCNPs and BMDC membranes. Overall, the composition of membrane proteins was mostly retained on the fabricated MCNPs compared to the BMDCs.

MCNPs can directly engage with immature and activated T cells to influence their response. The developed MCNPs leveraged the surface molecules found on professional APCs to interact with T cells. With a CD4 transgenic T cell receptor specific to BALB/c MHC II on dendritic cells, 4C mice were used to model the APCs/T cell direct allogeneic interaction.

Proliferation was increased compared to both control groups, PLG NPs alone and C57BL/6 MCNPs, indicating the allo-specificity between 4C T cells and MCNPs. A similar proliferation was found in the MCNP's co-culture with wild-type C57BL/6 T cells. The interaction with activated T cells was also investigated, with a reduction in both T-cell proliferation and pro-inflammatory cytokine levels (IFN-γ and IL-2), and an increase in anti-inflammatory cytokines (TGF-β and IL-10) observed with MCNP co-culture. TGF-β broadly impedes T cell activation by inhibiting TCR signaling, which hinders IL-2 production. In vitro-activated T cells are highly dependent on IL-2, and the lower IL-2 level with MCNP co-culture may explain the lower T cell viability that was observed. The cytokine levels may also contribute to the differential response between MCNPs and BMDCs. The MCNPs are not able to produce cytokines, and thus cannot amplify the response in the way that BMDCs can. Collectively, the developed MCNPs have capability to directly interact with both naïve and activated T cells and modify T-cell cytokine activity.

As shown herein, MCNPs can be readily manipulated for controlling their interactions with T cells. The presentation of antigen can be tuned, with antigen loading either pre-or post-manufacture, to induce CD4 T cell proliferation. MCNPs were also engineered by modifying cell culture protocols or functionalizing MCNPs post-production. Activating the immature BMDCs into a population with high expressions in both MHC class II and co-stimulatory molecules can induce higher T cell proliferation relative to a more regulatory DC culture. The cytokine levels of anti-inflammatory TGF-β and pro-inflammatory IL-2 indicated the activated BMDC phenotype. MCNPs were also functionalized chemically for conjugation of ligands that can direct T cell responses. MCNPs were modified with biotin post-fabrication, allowing for attachment of streptavidin-attached proteins. SA modified immunomodulators, such as SA-FasL or SA-PD-L1 (Batra, L. et al. Localized Immunomodulation with PD-L1 Results in Sustained Survival and Function of Allogeneic Islets without Chronic Immunosuppression. J. Immunol. 204, 2840-2851 (2020)) can be immobilized for directing T cell responses.

In conclusion, the membrane-coated nanoparticles provided herein can directly interact with T cells to influence responses. MCNPs, which present surface molecules that interact with T cells, can be modified pre-or post-fabrication for tuning the presentation of surface molecule modifications and antigen loading. MCNPs also demonstrated to function similarly to their BMDC counterparts in terms of interacting with T cells and inducing cytokine secretion of T cells. These MCNPs provide an off-the-shelf product that can be applied to modulate immune responses.

### Methods

Particle Synthesis: RESOMER Poly Lactic-co-Glycolic Acid (PLGA) (0.15 Low MW) (Evonik, Germany), polyvinyl alcohol (PVA) (Sigma-Aldrich, MO), sonicator (Cole-Parmer, IL)

Cells: Mouse A20 cells (ATCC, VA), RPMI 1640 (Gibco, NY), 10% heat-inactivated fetal bovine serum (VWR, PA), 0.05 mM 2-mercaptoethanol (Sigma-Aldrich, MO). Bone marrow derived dendritic cells (BMDC) were isolated from female BALB/c mice (The Jackson Laboratory, ME), and cultured with R10 media (RPMI 1640 (Gibco, NY), 10% heat-inactivated fetal bovine serum (VWR, PA), 0.05 mM 2-mercaptoethanol (Sigma-Aldrich, MO), 1% Penicillin-Streptomycin (Gibco, NY), 2 mM L-glutamine (Gibco, NY)), Recombinant murine granulocyte-macrophage colony-stimulating factor (GM-CSF) (PeproTech US, NJ), Dexamethasone (Sigma-Aldrich, MO), Lipopolysaccharide (LPS) isolated from E. coli (Sigma-Aldrich, MO), Recombinant Human TNF-α (PeproTech US, NJ)

Membrane extraction and coating: Hypotonic solution (DI water, 20 mM Tris-HCl (pH=7.5) (Fisher Scientific, NH), 10 mM potassium chloride (Sigma-Aldrich, MO), 2 mM magnesium chloride (Sigma-Aldrich, MO), and 1× Halt protease inhibitor (Fisher Scientific, NH), 1 mL dounce tissue grinder (VWR, PA), extruder set with holder/heating block (Avanti, AL), polycarbonate filters 0.8 μm 19 mm or 1 μm (Avanti, AL).

In vitro assays: 4C mice (PMID: 18212630) bred at the University of Missouri, Columbia, OT-II mice (The Jackson Laboratory, ME), C57BL/6J mice (The Jackson Laboratory, ME), mouse CD4⁺ T cell isolation kit (Miltenyi Biotec, Germany), Dynabeads Mouse T-Activator CD3/CD28 for T-Cell Expansion and Activation (Gibco, NY), EZ-Link Sulfo-NHS-LC-Biotin (Thermo Scientific, MA), streptavidin-Fas Ligand protein was expressed in Drosophila S2 cells, purified, and characterized as previously reported (Biomaterials. 2019 February: 192:271-281. doi:10.1016/j.biomaterials.2018.11.015. Epub 2018 Nov. 13.)

Flow cytometry antibodies (BioLegend, CA if not otherwise noted): PE anti-mouse CD86 Antibody, Brilliant Violet 421 anti-mouse CD80 Antibody, FITC anti-mouse CD4 Antibody, PE anti-mouse CD25 Antibody, FITC anti-mouse I-A/I-E Antibody, APC anti-mouse CD11c Antibody, Pacific Blue anti-mouse CD40 Antibody, Brilliant Violet 605 anti-mouse CD274 (B7-H1, PD-L1) Antibody, PE anti-mouse CD275 (B7-H2, B7-RP1, ICOS Ligand) Antibody, DAPI, CellTrace far red cell proliferation kit (Invitrogen, MA), Dead Cell Apoptosis Kits with Annexin V for Flow Cytometry (Invitrogen, MA)

PLG Nanoparticle Synthesis: 50 mg of RESOMER PLGA (50:50, 0.15 Low MW) was dissolved in 1 ml of dichloromethane. The organic solution was added to 5 ml of 1% PVA solution. The solution was sonicated for 30 seconds at 100% amplitude. The sonicated mixture was poured to 100 ml of 0.5% PVA and was allowed to evaporate. Following evaporation of dichloromethane after 12 hours, the solution was passed through a 40-micron filter and collected by centrifugation at 7500 g. The PLGA nanoparticles were washed three time by DI water for the membrane extraction. Nanoparticles of different sizes were developed and tested, including 300 nm and 400 nm NP sizes.

BMDC culture for regulatory DC: Bone marrow stem cells were harvested from BALB/c mice. Briefly, R10 media replenished by 20 ng/ml GM-CSF was added to culture on Day 0, 3, 6, and 8. Starting day 6, 10-7 M Dexamethasone and 1 μg/mL LPS were added with R10 media. Regulatory DC were ready by Day 9 and confirmed by flow cytometry.

Cell membrane extraction: A20 cell or BMDCs were harvested and resuspended in 1 ml of hypotonic solution. The cell mixture was disrupted using a dounce tissue grinder with a tight-fitting pestle for 50 passes before spinning down at 3200 g for 5 minutes. The supernatant was collected, and the cell pellet was resuspended in 1 ml of hypotonic solution for another round of homogenization. The homogenization was repeated three times before all the collected supernatants were spun at 20,000 g for 20 minutes. The supernatant was then spun at 100,000 g for 1 hour using an ultracentrifuge to isolate the cell membrane.

Nanoparticle cell membrane coating: To prepare cell membrane vesicles, membrane material derived as described above was physically extruded through a polycarbonate filter for 11 passes in an extruder set. The resulting vesicles were then coated onto the PLGA nanoparticles by co-extruding vesicles and cores through a polycarbonate filter. The final MCNPs were separated from the excess membrane vesicles by centrifugation at 10,000×g for 5 min. For certain experiments conducted herein, an 800 nm polycarbonate filter was used for extrusion and co-extrusion. For other experiments conducted herein, a 1 micron polycarbonate filter was used for extrusion and co-extrusion.

MCNP Characterization: Size and (-potential were measured by using Dynamic Light Scattering (MALVERN ZETASIZER NANO ZSP) in PBS solution at 25° C.; Nanoparticle surface molecules by flow cytometry for CD11c, MHCII, CD86, CD80, CD40, CD275 (ICOSL), CD274 (PD-L1)

CD4 T cell purification and staining: Naive CD4 T cells from 4C mice or C57BL/6J were extracted from the spleen by using a mouse CD4+ T cell isolation kit per manufacturer's instructions. The naive CD4 T cells were stained by CellTrace Far Red (CFSE).

MCNPs with naïve C57BL/6 or naïve 4C T cells: BMDC coated MCNPs were co-cultured with T cell subpopulations from different murine sources. The final concentration of MCNPs was 200 ug/ml and T cell population was 100,000/well for all conditions The stained naive CD4 T cells were co-cultured under varied experimental conditions for 3 days before the flow cytometry analysis.

MCNPs with activated C57BL/6T cells: Naïve CD4 T cells were activated by Dynabeads per manufacturer's instructions. The activation was confirmed by the dilution of CSFE signal and Dynabeads were removed by magnet before co-culture. The activated T cells were co-cultured with under varied experimental conditions for 3 days before the flow cytometry analysis. Dead Cell Apoptosis Kit was then used for apoptosis testing.

MCNP surface engineering and co-culture with OT-II naïve T cells: OVA peptide was presented by MCNP by two methods. 1. OVA peptide 323-339 (2ug/mL) was added to the BMDC culture on Day 9 and the OVA323-339-loaded BMDC membrane was harvested by Day 11 for MCNP fabrication. 2. OVA 323-339 was co-cultured with blank BMDC MCNP for 4 hours for uptake. Similar to the abovementioned methods, the CFSE stained naïve CD4 T cells from OT-II mice were co-cultured under varied experimental conditions for 3 days before the flow cytometry analysis.

Biotinylation of MCNPs: MCNPs were incubated in 5 mM EZ-Link Sulfo-NHS-LC-Biotin solution at 20° C. for 30 minutes. After incubation, MCNPs were washed three times in PBS to remove any unbound biotin. MCNPs were then incubated in PBS containing SA-FasL protein (400 ng SA-FasL/200 μl PBS) at 20° C. for 30 minutes. MCNPs were washed three times again in PBS to remove any unbound protein before co-culture. A20 cells (100,000 cells/well) was co-cultured with 200 ug/ml MCNPs for 24 hours before next step. Cells were stained by Dead Cell Apoptosis Kits with Annexin V per manufacturer's instructions before testing for apoptosis.

ELISA assays for cytokine concentrations: The co-culture supernatant was collected and cytokine concentrations were measured using corresponding ELISA kit.

1. A membrane-coated nanoparticle, comprising a nanoparticle core coated with a cell membrane from a cell that contains antigens.

2. The membrane-coated nanoparticle of claim 1, wherein the cell that contains antigens is an antigen presenting cell or a stem cell.

3. The membrane-coated nanoparticle of claim 2, wherein the antigen presenting cell is a regulatory antigen presenting cell or a tolerogenic antigen presenting cell.

4. The membrane-coated nanoparticle of claim 2, wherein the antigen presenting cell is a B-cell, a dendritic cell, a regulatory dendritic cell, a bone marrow-derived regulatory dendritic cell, or an activated bone marrow-derived regulatory dendritic cell.

5. (canceled)

6. (canceled)

7. (canceled)

8. (canceled)

9. The membrane-coated nanoparticle of claim 1, wherein the nanoparticle core comprises poly(lactide-co-glycolide) (PLG).

10. The membrane-coated nanoparticle of claim 1, wherein the membrane-coated nanoparticle expresses three or more surface markers selected from CD11c, MHC-II, CD80, CD86, CD40, PD-L1, and ICOSL.

11. (canceled)

12. (canceled)

13. The membrane-coated nanoparticle of claim 1, wherein the membrane-coated nanoparticle is substantially spherical in shape and has an average diameter of about 300 to about 800 nm.

14. (canceled)

15. The membrane-coated nanoparticle of claim 1, further comprising one or more immunomodulators conjugated to the cell membrane.

16. A method of producing a membrane-coated nanoparticle, the method comprising:
a) extracting a cell membrane from a cell that contains antigens; and
b) coating a nanoparticle core with the cell membrane.

17. The method of claim 16, wherein the nanoparticle core comprises poly(lactide-co-glycolide) (PLG).

18. The method of claim 16, wherein the cell that contains antigens is an antigen presenting cell or a stem cell.

19. The method of claim 18, wherein the cell that contains antigens is a regulatory antigen presenting cell or a tolerogenic antigen presenting cell.

20. The method of claim 18, wherein the antigen presenting cell is a B-cell, a dendritic cell, a regulatory dendritic cell, a bone marrow-derived regulatory dendritic cell, or an activated bone marrow-derived regulatory dendritic cell.

21. (canceled)

22. (canceled)

23. (canceled)

24. (canceled)

25. The method of claim 16, wherein the nanoparticle core is substantially spherical in shape and has an average diameter of about 100 nm to about 700 nm.

26. (canceled)

27. The method of claim 25, wherein the nanoparticle core has an average diameter of about 200 nm to about 400 nm.

28. (canceled)

29. The method of claim 16, wherein extracting the cell membrane comprises lysing the cell that contains antigens to obtain the cell membrane.

30. The method of claim 29, wherein extracting the cell membrane further comprises extruding the cell membrane through a filter having a pore size of about 600 nm to about 1400 nm.

31. (canceled)

32. (canceled)

33. The method of claim 16, wherein coating the nanoparticle core with the cell membrane comprises co-extruding the extracted cell membrane and the nanoparticle core through a filter.

34. The method of claim 33, wherein the filter has a pore size of about 600 nm to about 1400 nm.

35. (canceled)

36. (canceled)

37. A method of modulating an immune response in a sample or a subject, comprising contacting the sample or the subject with the membrane-coated nanoparticle of claim 1.

38. (canceled)

39. (canceled)

40. (canceled)

41. (canceled)

42. (canceled)
